(19) United States
(12) Patent Application Publication
(10) Pub. No.: US 2014/0162902 A1
(43) Pub. Date: Jun. 12, 2014

(54) Cell-Based Materials and Methods for Defining Pharmacogenetic Differences in Drug Metabolism

(71) Applicants: Joshua LaBaer, Chandler, AZ (US); Brianne Petritis, Phoenix, AZ (US)

(72) Inventors: Joshua LaBaer, Chandler, AZ (US); Brianne Petritis, Phoenix, AZ (US)

(73) Assignee: Arizona Board of Regents of behalf of Arizona State University, Scottsdale, AZ (US)

(21) Appl. No.: 14/236,836

(22) PCT Filed: Jul. 27, 2012
(86) PCT No.: PCT/US12/48725

§ 371 Date: Feb. 3, 2014

Related U.S. Application Data
(60) Provisional application No. 61/515,012, filed on Aug. 4, 2011.

(51) Int. Cl. C12Q 1/26

(52) U.S. Cl. CPC C12Q 1/26
USPC 506/11; 435/25; 506/14; 435/6.11

(57) ABSTRACT

Cell lines harboring a range of polymorphisms using recombinant cytochrome P450 or other chemical-metabolizing enzymes in a parent cell line that is minimally expressing or devoid of its own cytochrome P450 protein or other chemical-metabolizing enzymes of interest can be placed into an array format to enable high throughput screening of one or more chemicals for CYP450 or other enzyme-dependent metabolism (FIG. 9). Processing of the cells can be automated, done en-masse through use of an array having a substrate upon which a plurality of cell lines with exogenous chemical-metabolizing enzymes are coupled, and both relative and quantitative metabolism rates determined using mass spectrometry over time. Thus, methods are disclosed to measure, for example, the effects of cytochrome P450 polymorphisms on clinical drug metabolism in a high throughput manner for drug development and genetically personalized diagnostics and treatment regimens.

### BACKGROUND OF THE INVENTION

Cytochrome CYP450 (CYP450) enzymes, such as CYP1A1, CYP1A2, CYP2A6, CYP2B6, CYP2C9/CYP2C10, CYP2C19, CYP2D6, CYP2E1, and CYP3A4, metabolize ˜80% of clinically-used drugs, as well as other exogenous chemicals to which humans are exposed. These enzymes are highly polymorphic in the human population, leading to a need for research on the effects that the polymorphisms have on metabolism of clinical drugs or other chemicals.

These polymorphisms can result in no enzyme activity, impaired activity, or altered activity. Those that decrease drug metabolism can cause a patient to suffer from drug toxicity since the drug substrate is built up over time in the system without being excreted. On the other hand, increased drug metabolism results in the drug having little therapeutic effect on the patient since the drug is excreted too rapidly. For example, 7-10% of women with breast cancer get little or no therapeutic benefit from the drug tamoxifen because their polymorphic CYP2d6 enzyme is unable to modify the ingested drug to its active form, thus increasing the chance of death or cancer recurrence. Although the effect of this specific CYP2D6 polymorphism is understood, there are hundreds of other CYP2D6 and CYP450 polymorphisms that are not understood in terms of their effects on tamoxifen or any other drug.

Chemicals may be activated or inactivated by the CYP450 enzymes, which will lead to different effects in the body than those described above depending on how a polymorphism(s) may affect CYP450 functionality. It is possible that polymorphisms may lead to new and unknown metabolites. These new metabolites could have direct or indirect effects on molecular processes, which could be detrimental or beneficial to the individual. It is possible that polymorphic CYP450 may preferentially favor the formation of certain metabolites over others.

To investigate such effects, current in vitro methods rely on cell components, such as endoplasmic reticulum fragments called microsomes. However, only the most common CYP450 enzyme isotypes have been examined due in part to the cumbersome process of microsomal enrichment. Moreover, microsome use may not represent in vivo metabolism as well as a cell-based assay since microsomes have limited phase II metabolic activity and a limited subset of interactions with other molecular events that occur in vivo (e.g., drug transporters).

### SUMMARY OF THE INVENTION

Embodiments disclosed herein relate to materials and methods of assessing a chemical, such as a drug, for CYP450 or other enzyme-dependent metabolism in a cell-based system.

In some embodiments, the methods include the steps of transfecting or transducing a cell line that is deficient or absent of CYP450 or another chemical-metabolizing enzyme expression with CYP450 DNA (or that of another chemical-metabolizing enzyme) that will allow the exogenously-introduced DNA to be properly transcribed and translated into protein. The transfected or transduced cell line is then assayed by detecting the level of the chemical and its metabolite(s) over time.

In other embodiments, the methods include the step of assaying one or more cell lines expressing one or more CYP450 isoforms by detecting a level of a drug and its metabolite(s) over time.

Yet other embodiments include analyzing the effect of polymorphisms of other chemical-metabolizing enzymes. For example, cytochrome P450 reductase, epoxide hydratase, glutathione S-transferase, etc.

In still other embodiments, cell lines that represent a selected range of polymorphisms using recombinant CYP450 enzymes in a parent cell line that is minimally expressing or devoid of its own CYP450 protein are created.

One or more of the cell lines described above may be placed into an array format to enable high throughput screening of one or more chemicals for CYP450-dependent metabolism or another chemical-metabolizing enzyme on one or more chemicals that are added to the cell line(s) either separately or simultaneously (i.e., a mixture of chemicals applied to one sample).

In other embodiments, one cell line may harbor more than one exogenously-introduced CYP450 isoforms and/or polymorphisms via transfection or transduction.

Processing of the chemical-treated cells can be automated and metabolism rates determined using mass spectrometry over time. Thus, methods are disclosed to measure, for example, the effects of cytochrome P450 polymorphisms on clinical drug metabolism in a high throughput manner for drug development and genetically personalized diagnostics and treatment regimens.

These and other aspects of the invention will be apparent upon reference to the following detailed description and figures. To that end, certain patent and other documents are cited herein. Each of these documents is hereby incorporated by reference in its entirety.

As used herein, the term “Cytochrome P450” or “P450” means the superfamily of enzymes that are officially abbreviated as CYP450.

### BRIEF DESCRIPTION OF THE DRAWINGS

FIG. 1 summarizes CYP450 background and function.

FIG. 2 depicts a table of xenobiotic metabolizing cytochrome P450 enzymes.

FIG. 3 schematically depicts pharmacogenetics of CYP450.

FIG. 4 summarizes CYP450 polymorphisms.

FIG. 5 is a table summarizing polymorphisms of select CYP450 enzymes.

FIG. 6 summarizes some CYP450 pharmacogentics methods.

FIG. 7 summarizes further CYP450 pharmacogentics methods.

FIG. 8 schematically depicts a high-throughput method of the invention using an array.

FIG. 9 summarizes CYP450 polymorphism effects.

FIG. 10 summarized an array used with methods of the invention.

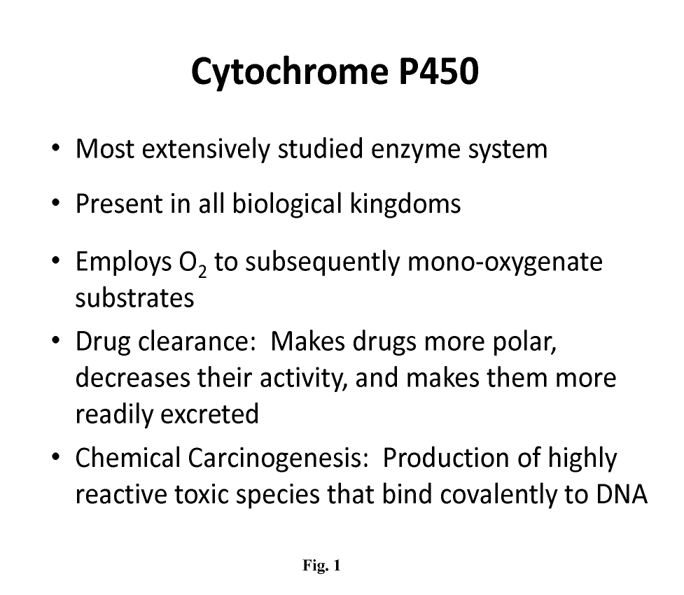

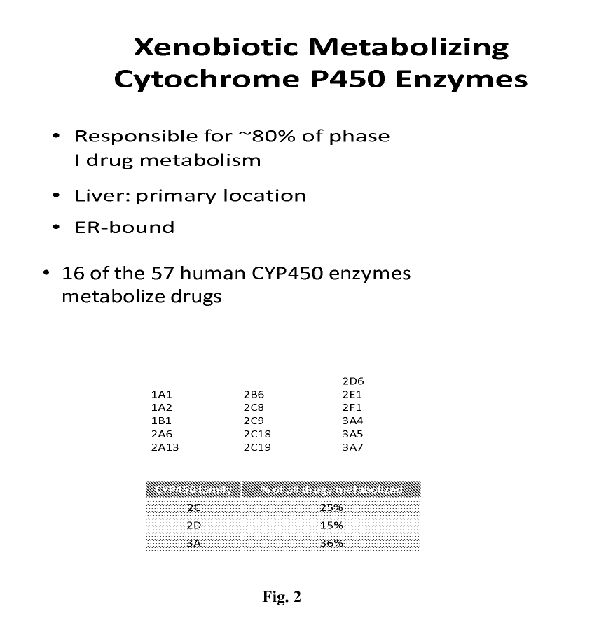

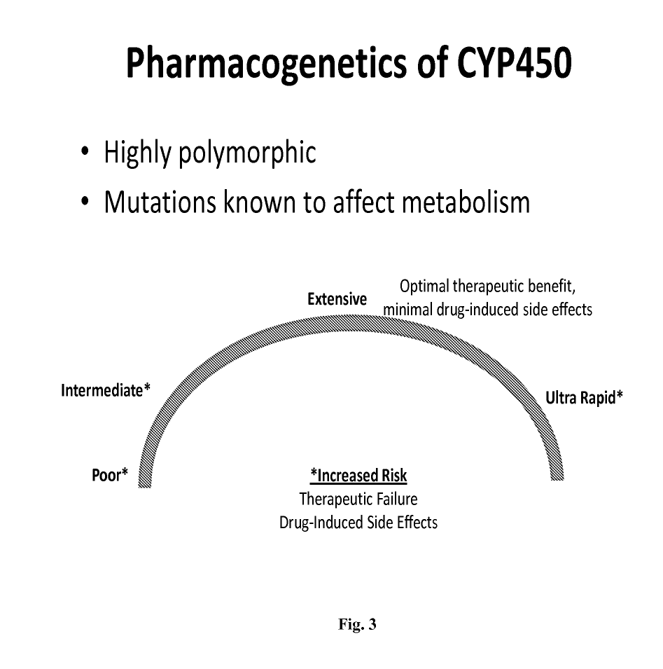

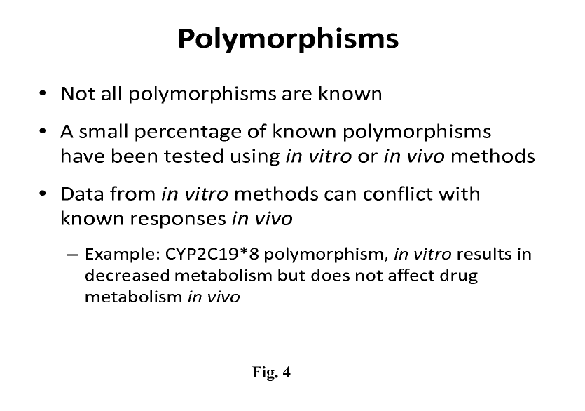

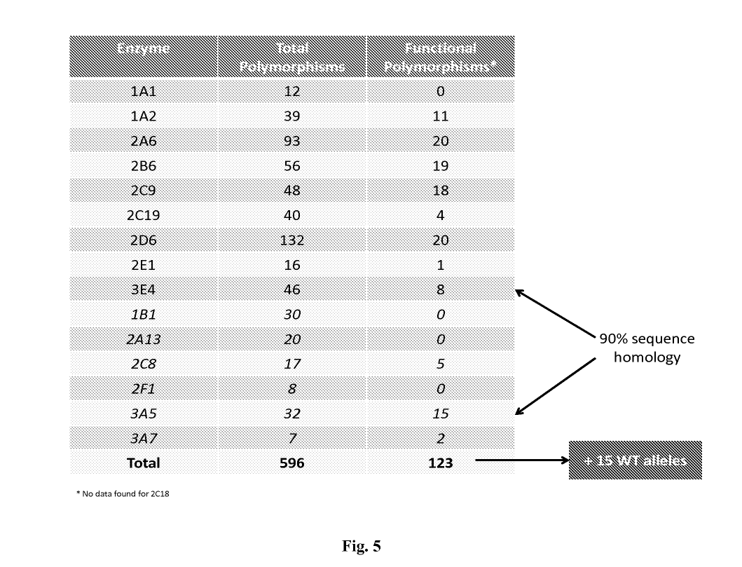

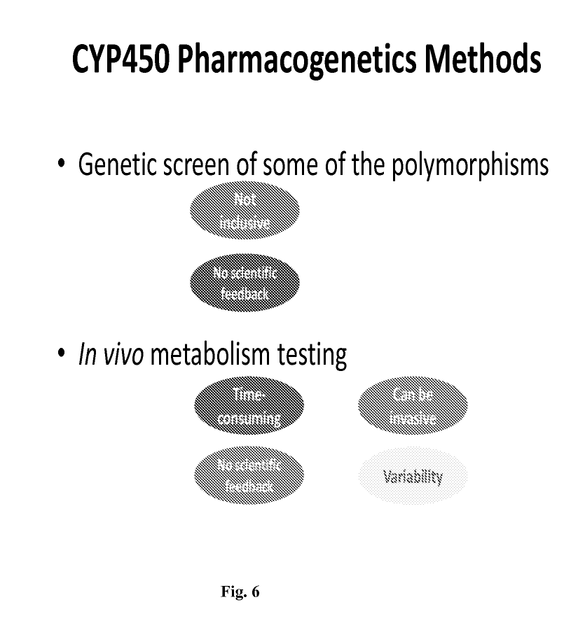

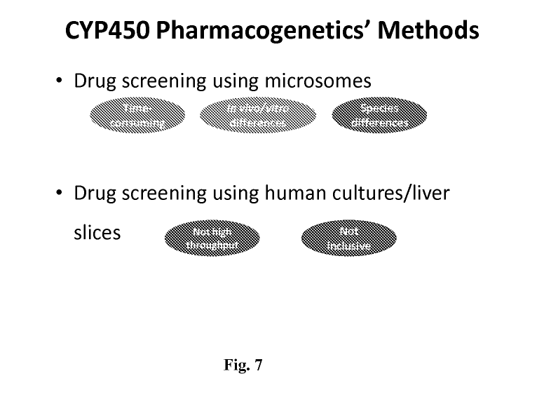

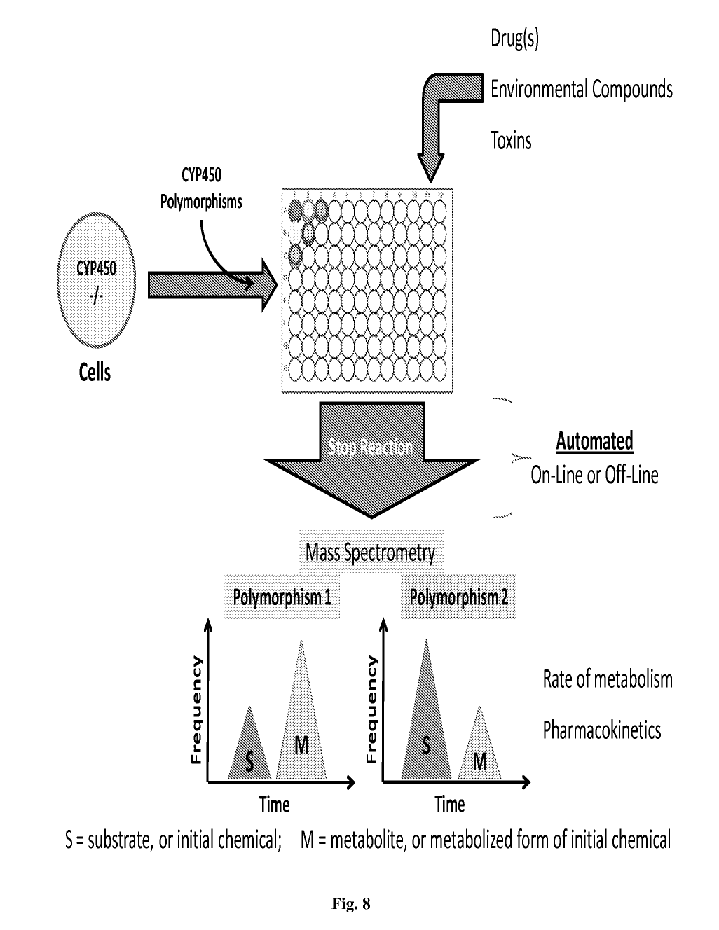

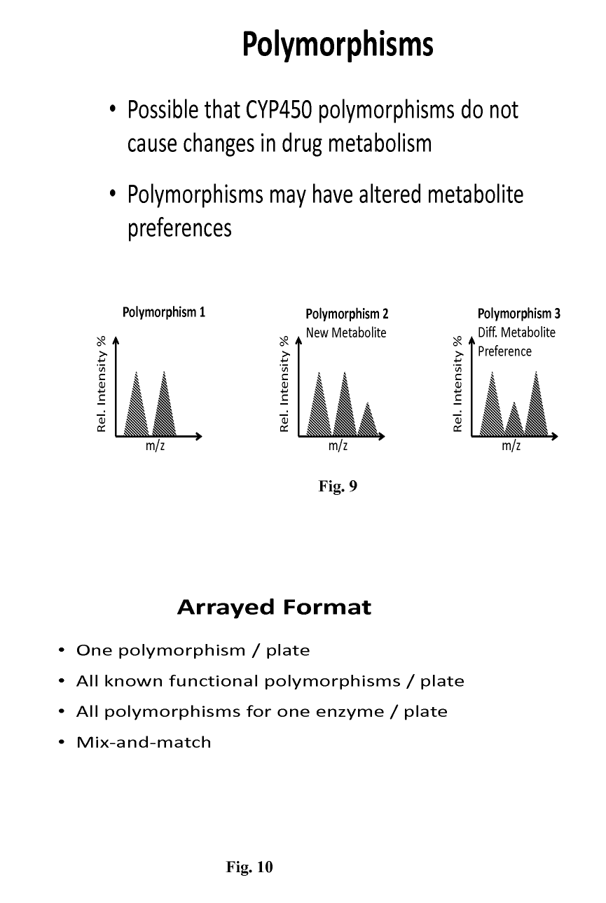

### DETAILED DESCRIPTION OF THE INVENTION

Embodiments of the inventions relate to the characterization of cytochrome P450 polymorphisms to provide, for example, drug response predictions specific to an individual's genetic makeup.

Thus, cell lines that represent the range of polymorphisms using recombinant cytochrome P450 enzymes in a parent cell line that is minimally expressing or devoid of its own cytochrome P450 protein-of interest are described. These cells can then be placed into an array format that will enable high throughput screening of drug metabolism. Processing of the cells can be automated and drug metabolism could be determined using mass spectrometry (e.g., mass spectrometry with selected reaction monitoring (SRM)).

Embodiments of the invention involve screening the effects of cytochrome P450 polymorphisms on clinical drug metabolism in a high throughput manner for drug development and personalized diagnostics. For example, methods of the invention can be used for investigating the effects of CYP450 polymorphisms on one drug at a time as well as multiple drugs at a time (i.e., drug-drug interactions). Moreover, methods of the invention can involve analyzing the effect of drug metabolism by multiple CYP450 polymorphisms and/or isoforms at one time to better understand the relationship between them.

Methods of the invention are applied toward characterizing human cytochrome P450 responses to other “chemicals,” such as environmental compounds or possible toxins (insecticides, herbicides, natural products, chemical exposures, etc.), which includes the effect of CYP450 polymorphisms on disease susceptibility, such as cancer and autoimmune disease.

Methods of the invention also involve creating and utilizing cell lines for xenobiotic metabolizing cytochrome P450 research, polymorphic recombinant cell assays, and drug screening. Genes encoding human recombinant xenobiotic metabolizing cytochrome P450 enzymes can be obtained, either from existing gene collections in academic labs, by production in the lab by molecular biological methods that are known in the art, or from commercial sources such as Open Biosystems from Thermo Fisher Scientific, Inc.

Cell lines that are minimally expressing or devoid of one or more endogenous CYP450 enzymes can be used for expression of recombinant polymorphic versions of the CYP540 enzymes, with cells being placed into an arrayed format and the chemical(s) of interest being added. Then cells can be processed and enzymatic activity determined by analyzing the compound substrate and metabolic product(s) (i.e., metabolite) using mass spectrometry or other means for measuring same, such as quantitative nuclear magnetic resonance or spectrophotometric methods that are known in the art.

Embodiments of the invention could also include cell lines that are minimally expressing or devoid of one or more endogenous drug-metabolizing enzymes, such as cytochrome P450 reductase, epoxide hydratase, and glutathione S-transferase. These cell lines can be used for expression of recombinant polymorphic versions of these enzymes, with cells being placed into an arrayed format and the chemical of interest being added. Then cells can be processed and enzymatic activity determined by analyzing the compound substrate and metabolic product(s) (i.e., metabolite) using mass spectrometry or other means for measuring same, such as quantitative nuclear magnetic resonance or spectrophotometric methods that are known in the art.

Methods described herein may include in vitro recombinant DNA techniques, synthetic techniques, and in vivo genetic recombination in a variety of expression vector/host systems. These methods are described in standard laboratory references, such as Sambrook, J. et al. Molecular Cloning, A Laboratory Manual, Cold Spring Harbor Press, Plainview, N.Y. (1989) and Ausubel, F. M. et al. Current Protocols In Molecular Biology, John Wiley & Sons, Inc., New York (2007).

In addition to those described above, other embodiments of the invention could employ surface plasmon resonance and polymorphic recombinant enzymes using a nucleic acid programmable protein array strategy that is further described in the U.S. Pat. No. 6,800,453 to better understand enzyme-drug and drug-drug binding kinetics in a high throughput fashion.

Still other embodiments of the invention could employ surface plasmon resonance or mass spectrometry and polymorphic recombinant enzymes using nanodiscs (i.e., a model membrane system to study membrane proteins) to better understand enzyme-drug and drug-drug binding kinetics in a high throughput fashion. See Denisov, I G et al. Cytochromes P450 in Nanodiscs. Biochimica et Biophysica Acta-Proteins and Proteomics 1814, 223-229 (2011); Bayburt, T H et al. Self-Assembly of Discoidal Phospholipid Biolayer Nanoparticles with Membrane Scaffold Proteins. Nano Letters 2, 853-856 (2002); and Denisov, I G et al. Directed Self-Assembly of Monodisperse Phospholipid Bilayer Nanodiscs with Controlled Size. Journal of American Chemical Society 126, 3477-3487 (2004).

The methods described herein can utilize polymorphisms in the CYP450 (or other chemical-metabolizing enzyme) coding region (i.e., translated portion of the nucleotide sequence), polymorphisms in the five prime and three prime untranslated regions (UTR) of CYP450 messenger RNA, and/or polymorphisms in the genomic DNA in and around the CYP450 functional gene structure, which may include introns, enhancers, promoters, inhibitors, etc.

The methods herein also could employ a combination of polymorphisms in the CYP450 (or other chemical-metabolizing enzyme) coding, untranslated regions, and functional gene structure.

No currently-available assay is known to provide a cell-based means for investigating the effect of cytochrome P450 polymorphisms at the enzyme level on the rate of drug metabolism in vitro. Thus, the methods of the invention provide a new and inventive variety of research tools and methods. Such tools and methods can include: (1) Creation or utilization of a CYP450-expressing cell line previously deficient in part or all of the xenobiotic-metabolizing cytochrome P450 enzyme activity; (2) analyzing the effect of cytochrome P450 reductase polymorphisms (POR) on drug metabolism through the use of recombinant technology (POR is an enzyme that enables electron transport to the xenobiotic-metabolizing CYP450 enzymes); (3) analyzing the effect of other chemical-metabolizing enzyme polymorphisms on metabolism; (4) analyzing the combinatorial effect of two or more cytochrome P450 enzymes and their polymorphisms on drug/compound metabolism; (5) analyzing drug-drug and enzyme-drug interactions using technology capable of determining binding kinetics; for example, surface plasmon resonance or mass spectrometry; (6) analyzing the effect of drug metabolism by multiple CYP450 polymorphisms and/or isoforms at one time to better understand the relationship between them.

### NON-LIMITING EXAMPLES

A method of screening a chemical for CYP450-dependent metabolism using a CYP450 deficient or absent cell line, such as Chinese hamster lung V79 or human hepatocellular carcinoma HepG2 obtained from American Type Culture Collection (ATCC). Exogenous human CYP450 DNA will be introduced into the cell lines via transfection or transduction according to well-established molecular biology procedures as outlined in standard laboratory references, such as those described above. This involves the use of circular DNA (i.e., plasmid) that includes the gene-of-interest as well as other elements necessary for the proper selection of transfected or transduced cells (e.g., antibiotic resistance) and expression of the gene-of-interest into protein (e.g., cytomegalovirus promoter). Polymorphic alleles are created using standard polymerase chain reaction procedures or from short DNA oligonucleotides purchased from commercial sources, such as Integrated DNA Technologies, Inc.

Once the transfected/transduced cell line(s) are applied to a high format array and adhered to the bottom, one or more chemicals are added to the cells. After a specific amount of time that is dependent on the chemical(s) and objective of the assay, the reaction (i.e., metabolism of the chemical) is stopped using an organic solvent (e.g., acetonitrile, methanol, etc.) at high concentrations (≧50%). The level of each chemical and its metabolite(s) over time are analyzed using mass spectrometry, which may include the use of on-line or off-line capillary electrophoresis, gas chromatography, liquid chromatography, direct infusion, and/or selected reaction monitoring (SRM).

The ratio of chemical to its metabolite(s) or between metabolites can be used to make an assessment of the relative rate of drug metabolism between CYP450 polymorphisms when the reaction time is the same across the CYP450 polymorphic cell lines. Alternatively, specific metabolic rate information could be obtained when a chemical(s) is added to the same cell line but the reaction time across the array is staggered.

A method of measuring a polymorphism of CYP450 enzyme for metabolic activity on a chemical, comprising the step of assaying a cell line that expresses a CYP450 isoform and has been contacted with the chemical for a level of the chemical and its metabolite(s) over time. The assaying step of the method can be performed through mass spectrometry.

Multiple CYP450 (and/or other chemical-metabolizing enzyme) isoforms can be assayed simultaneously in the methods. For example, multiple alleles of CYP450 can be expressed in one cell line or two or more cell lines expressing different isoforms can be utilized on the same array. Multiple chemicals also can be assayed simultaneously in the methods.

The methods described herein can be utilized for a personalized drug dosing regime, for example, by comparing a CYP450 genotype from a sample of a patient to a result obtained with the drug and on a cell line that expresses the human CYP450 enzyme(s) of the patient's genotype.

The methods described herein can be utilized for screening a patient for metabolism of a drug, for example, by obtaining a genotype of a CYP450 enzyme from a sample of DNA-containing material for that patient and comparing the genotype to a metabolism rate measured for that drug.

The methods described herein can be utilized for measuring a rate of CYP450-dependent metabolism for a chemical by, for example, assaying a cell line expressing a CYP450 allele and treated with that chemical for a level of the chemical and its metabolite(s) over time.

The methods described herein can be utilized for measuring an effect of a CYP450 polymorphism in a cell line on metabolism of a chemical by, for example, measuring a level of that chemical and its metabolite(s) in the cell line over time.

The methods described herein can be utilized by a pharmaceutical company developing drugs to determine if its candidate compounds will be metabolized differently in individuals harboring different polymorphisms of CYP450, thus enabling the companies to take one of several actions:
a. Utilize structure activity relationships (SAR) and medicinal chemistry coupled with assay methods described herein to develop alternative forms of the compound that are both effective and not subject to alternate metabolism by different CYP450 polymorphisms;
b. Develop a different dosing strategy for the drug based on the results of studies using assay methods described herein and clinical trials that can be used in individuals with different genetic backgrounds;
c. Develop alternate compounds for use in individuals with different CYP450 backgrounds;
d. Develop a drug labeling process that indicates the relative risks and benefits of taking this compound in individuals with affected genetic backgrounds.

The materials and methods described above are not intended to be limited to the embodiments and examples described herein.

1. A method for measuring a chemical-metabolizing enzyme for a rate of metabolic activity, comprising the step of assaying a cell line that expresses an exogenous chemical-metabolizing enzyme and that has been contacted with a chemical to determine the level of said chemical and one or more of its metabolites over time.

2. The method of claim 1, wherein said cell line is devoid of endogenous activity for said exogenous chemical-metabolizing enzyme.

3. The method of claim 1, wherein said cell line is deficient of endogenous activity for said exogenous chemical-metabolizing enzyme such that the introduced chemical-metabolizing enzyme is expressed at higher levels than that expressed endogenously.

4. The method of claim 1, wherein at least two cell lines, polymorphisms of said chemical-metabolizing enzyme, chemicals, or a combination thereof are assayed simultaneously.

5. A method of screening a chemical for CYP450-dependent metabolism, comprising the steps of:
transfecting or transducing a cell line that is effectively devoid of one or more endogenous P450 enzymes of interest with DNA encoding and expressing one or more of said P450 enzymes; and
assaying said transfected or transduced cell line by detecting a level of said chemical and one or more of its metabolites over time.

6. The method of claim 5, wherein said assaying is performed through mass spectrometry.

7. The method of claim 5, wherein said assaying is performed through mass spectrometry using selected reaction monitoring (SRM).

8. The method of claim 5, wherein said DNA is human DNA.

9. The method of claim 5, wherein at least two cell lines, polymorphisms of said P450 enzymes, chemicals, or a combination thereof are assayed simultaneously.

10. A method of measuring a CYP450 enzyme for a rate of metabolic activity, comprising the step of assaying a cell line that expresses a CYP450 isoform and that has been contacted with said chemical to determine the level of said chemical and its metabolite(s) over time.

11. The method of claim 10, wherein said assaying is performed through mass spectrometry.

12. The method of claim 10, wherein said assaying is performed through mass spectrometry using selected reaction monitoring (SRM).

13. The method of claim 10, wherein said CYP450 isoform is human.

14. The method of claim 10, wherein said cell line is a CYP450 deficient or absent cell line with CYP450 DNA encoding and expressing said isoform being transfected or transduced into said cell line such that the introduced P450 isoform is expressed at higher levels than that expressed endogenously.

15. The method of claim 10, wherein at least two cell lines, isoforms, chemicals, or a combination thereof are assayed simultaneously.

16. A personalized drug dosing regime, comprising the step of comparing a CYP450 genotype determined from a sample of a patient to a rate of metabolism obtained according to the method of claim 10 for said drug and said genotype.

17. A method of screening a patient for metabolism of a drug, comprising the steps of obtaining a genotype of a CYP450 enzyme for said patient and comparing said genotype to a rate of metabolism obtained according to the method of claim 10 for said drug.

18. A method for characterizing the relative effect of a CYP450 polymorphism in a cell line on metabolism of a chemical, comprising measuring a level of said chemical and one or more of its metabolites in said cell line over time when compared to another CYP450 polymorphism.

19. The method of claim 18, wherein at least two cell lines are assayed simultaneously for said level of said chemical and its metabolite(s).

20. An array comprising a substrate to which a plurality of cell lines that express an exogenous chemical-metabolizing enzyme are coupled.
